(19) United States
(12) Patent Application Publication
(10) Pub. No.: US 2026/0112355 A1
(43) Pub. Date: Apr. 23, 2026

(54) SYSTEM

(71) Applicant: SoftBank Group Corp., Tokyo (JP)

(72) Inventor: Kazuaki TOBA, Tokyo (JP)

(21) Appl. No.: 19/353,231

(22) Filed: Oct. 8, 2025

(30) Foreign Application Priority Data
Oct. 18, 2024 (JP) 2024-183689

(51) Int. Cl. G10L 13/027; G06V 20/40; G10L 13/08; G10L 15/26; G10L 25/57; G10L 25/66; G16H 40/20

(52) U.S. Cl. CPC G10L 13/027; G06V 20/47; G10L 13/08; G10L 15/26; G10L 25/57; G10L 25/66; G16H 40/20; G06V 2201/03

(57) ABSTRACT

The system according to the embodiment includes a reception unit, an analysis unit, a generation unit, a reading unit, and a formatting unit. The reception unit records a video. The analysis unit analyzes the video recorded by the reception unit. The generation unit generates a summary based on the video analyzed by the analysis unit. The reading unit reads aloud the summary generated by the generation unit. The formatting unit launches a medical examination format based on the summary generated by the generation unit.

### CROSS-REFERENCE TO RELATED APPLICATION

The present application claims priority to and incorporates by reference the entire contents of Japanese Patent Application No. 2024-183689 filed in Japan on Oct. 18, 2024.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, including: receiving a user utterance, adding the user utterance to a prompt containing instructions related to the character of the chatbot, encoding the prompt, inputting the encoded prompt into a language model, and generating a chatbot utterance in response to the user utterance.

In conventional technology, it is difficult for doctors to quickly and accurately understand videos recorded by patients, which may lead to a decrease in the efficiency of medical examinations.

### SUMMARY OF THE INVENTION

The system according to the embodiment includes a reception unit, an analysis unit, a generation unit, a reading unit, and a formatting unit. The reception unit records a video. The analysis unit analyzes the video recorded by the reception unit. The generation unit generates a summary based on the video analyzed by the analysis unit. The reading unit reads aloud the summary generated by the generation unit. The formatting unit launches a medical examination format based on the summary generated by the generation unit.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor with a sign (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) with a sign is a memory where information is temporarily stored and used as a work memory by the processor.

In the following embodiments, a storage with a sign is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) with a sign is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

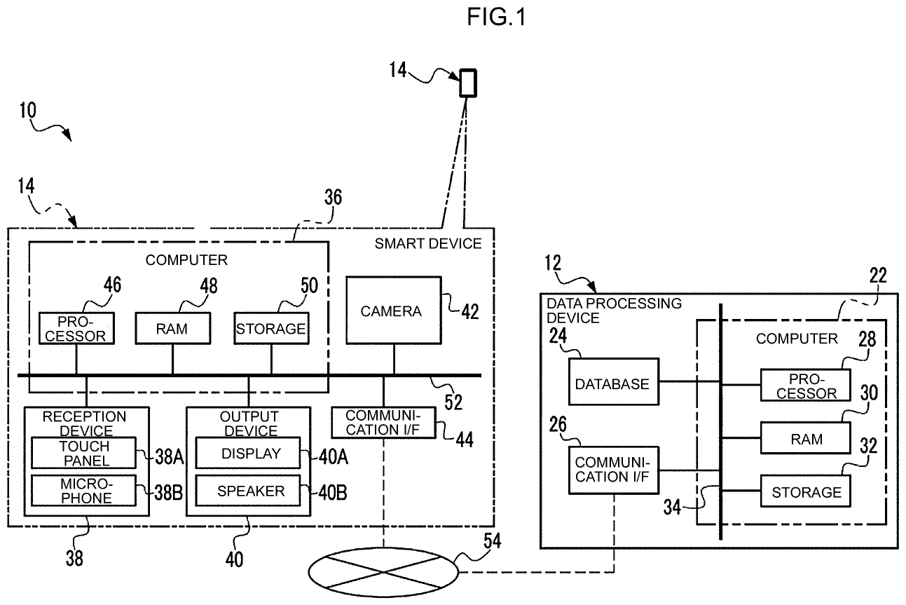

FIG. 1 shows an example configuration of a data processing system 10 according to the first embodiment.

As shown in FIG. 1, the data processing system 10 includes a data processing device 12 and a smart device 14. An example of the data processing device 12 is a server.

The data processing device 12 includes a computer 22, a database 24, and a communication I/F 26. The computer 22 includes a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 includes a computer 36, a reception device 38, an output device 40, a camera 42, and a communication I/F 44. The computer 36 includes a processor 46, RAM 48, and storage 50. The processor 46, RAM 48, and storage 50 are connected to a bus 52. The reception device 38, output device 40, and camera 42 are also connected to the bus 52.

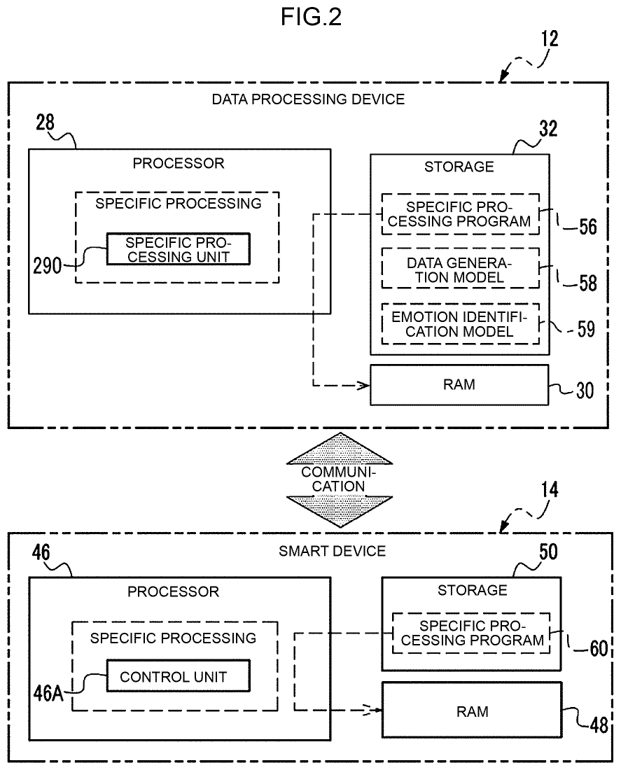

The reception device 38 includes a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 includes a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example 1 of Embodiment

The AI system according to the embodiment of the present invention is a system that summarizes videos recorded by a patient or a related person into content that is easy for a doctor to understand and acts as a bridge. In this AI system, the patient or a related person records the affected area while talking about symptoms. For example, symptoms such as “dull pain,” “nausea,” or “faintness” are recorded. In addition, items related to symptoms are recorded, such as complexion, condition of rashes, tremors, vomit, urine, etc. Next, in the case of an injury, the condition of the affected area and the object of the injury are recorded, such as the stairs that were fallen down, the ball that hit, or the insect that bit. These videos are analyzed by AI before arriving at the hospital and summarized for the doctor. Since the patient may not be able to speak after arrival, a summary reading function is also provided. Furthermore, as a second phase, it is also possible to have the medical examination format launched on the hospital side before the patient's arrival via communication. This AI system is particularly targeted at foreigners who are not proficient in Japanese, inbound tourists, young people who cannot explain their symptoms, elderly people with cognitive decline, and patients with severe symptoms who cannot speak. As a result, it is no longer necessary to explain verbally one-on-one to the doctor, and accurate information can be provided even if the memory is vague. In addition, the doctor can easily grasp the situation at the time of occurrence and eliminate language barriers and differences in nuance. This system bridges the person who knows the situation at the time of occurrence (the patient) and the person who provides treatment (the doctor), thereby eliminating recognition discrepancies. It is also possible to fill the time gap between occurrence and treatment. Furthermore, generative AI technologies such as summarization, translation, video analysis, emotion analysis, and language analysis are utilized. As a result, the AI system can summarize the patient's video into content that is easy for the doctor to understand and act as a bridge.

The AI system according to the embodiment includes a reception unit, an analysis unit, a generation unit, a reading unit, and a formatting unit. The reception unit records videos recorded by the patient or a related person. The videos recorded by the patient or a related person include, for example, videos in which the condition of the affected area or symptoms are described. The reception unit can record videos using, for example, a smartphone or tablet. The reception unit also has a function to upload the recorded videos to the cloud. The analysis unit analyzes the videos recorded by the reception unit. The analysis unit analyzes, for example, the content of the videos and extracts symptoms and the condition of the affected area. The analysis unit can analyze the content of the videos using AI. The generation unit generates a summary based on the videos analyzed by the analysis unit. The generation unit generates the summary using generative AI. The generation unit, for example, summarizes the content of the videos and converts it into a format that is easy for the doctor to understand. The reading unit reads aloud the summary generated by the generation unit. The reading unit can read aloud the summary using AI. The formatting unit launches a medical examination format based on the summary generated by the generation unit. The formatting unit can automatically generate the medical examination format using AI. Thus, the AI system according to the embodiment can summarize the patient's video into content that is easy for the doctor to understand and act as a bridge.

The reception unit records videos recorded by the patient or a related person. The videos recorded by the patient or a related person include, for example, videos in which the condition of the affected area or symptoms are described. The reception unit can record videos using, for example, a smartphone or tablet. Specifically, the patient uses the camera of a smartphone to shoot detailed images of the affected area and records a video in which the symptoms, degree of pain, and onset time are verbally explained. As a result, even if the doctor cannot examine the patient directly, detailed information can be provided. The reception unit also has a function to upload the recorded videos to the cloud. Uploading to the cloud is performed in an encrypted manner to ensure security and protect the patient's privacy. Furthermore, the reception unit also has a function to notify when the video upload is complete, allowing the patient or related person to check the transmission status of the video. Thus, the reception unit can efficiently and securely collect the patient's videos and quickly move to the next analysis step.

The analysis unit analyzes the videos recorded by the reception unit. The analysis unit analyzes, for example, the content of the videos and extracts symptoms and the condition of the affected area. The analysis unit can analyze the content of the videos using AI. Specifically, the AI uses image recognition technology for each frame of the video to analyze the condition of the affected area and detect changes in color, degree of swelling, presence or absence of bleeding, etc. In addition, speech recognition technology is used to convert the patient's explanation into text and extract information such as details of symptoms, onset time, and degree of pain. Furthermore, natural language processing technology is used to analyze the extracted text data and identify important keywords and phrases. As a result, the analysis unit can integrate visual and audio information obtained from the video and grasp the patient's symptoms and the condition of the affected area in detail. The analysis unit provides important data for the doctor to make a diagnosis based on this information.

The generation unit generates a summary based on the videos analyzed by the analysis unit. The generation unit generates the summary using generative AI. Specifically, the generative AI generates a summary in a format that is easy for the doctor to understand based on the text data and image data provided by the analysis unit. For example, the generative AI concisely summarizes the patient's symptoms and the condition of the affected area and emphasizes important points. In addition, the generative AI adjusts the summary to preferentially include information necessary for the doctor to make a diagnosis. As a result, the generation unit provides support for the doctor to quickly grasp the patient's condition and make an appropriate diagnosis. Furthermore, the generation unit can refer to past diagnostic data and medical literature to improve the accuracy of the summary and generate the optimal summary. Thus, the generation unit can always provide high-quality summaries reflecting the latest medical knowledge.

The reading unit reads aloud the summary generated by the generation unit. The reading unit can read aloud the summary using AI. Specifically, the reading unit uses speech synthesis technology to read aloud the generated summary in a natural voice. The speech synthesis technology converts text data into audio data and can read aloud with natural intonation and pronunciation. As a result, the doctor can not only visually check the summary but also listen to it by voice, thereby improving the efficiency of diagnosis. Furthermore, the reading unit has functions to adjust the reading speed and volume, allowing customization according to the doctor's preferences. Thus, the reading unit supports the doctor in obtaining information in the most efficient way. In addition, the reading unit can also support multiple languages and can read aloud the summary in different languages, making it useful in international medical settings.

The formatting unit launches a medical examination format based on the summary generated by the generation unit. The formatting unit can automatically generate the medical examination format using AI. Specifically, the formatting unit organizes the information necessary for the examination based on the generated summary and converts it into a standardized format. For example, the formatting unit automatically creates a medical examination format including the patient's basic information, details of symptoms, condition of the affected area, past medical history, etc. As a result, the doctor can save the trouble of manually creating the medical examination format and concentrate on diagnosis. Furthermore, the formatting unit can link the medical examination format with the electronic medical record system and quickly record the examination results. This improves the efficiency of medical examinations and reduces the workload in medical settings. In addition, the formatting unit can also customize the medical examination format and flexibly respond to the doctor's needs. Thus, the formatting unit provides support for the doctor to perform optimal examinations and can improve the treatment effect for the patient.

The reception unit can record a video in which a patient or a related person talks about symptoms while recording the affected area. For example, the reception unit records the patient talking about symptoms such as “dull pain,” “nausea,” or “faintness” while recording the affected area. In addition, the reception unit can also record items related to symptoms, such as complexion, condition of rashes, tremors, vomit, urine, etc. As a result, the reception unit can record a video in which a patient or a related person talks about symptoms while recording the affected area. Some or all of the above-described processing in the reception unit may be performed using AI or may be performed without using AI. For example, the reception unit can use speech recognition technology to convert the content spoken by the patient into text and store it together with the recorded content.

The reception unit can record items related to symptoms. For example, the reception unit records the patient's complexion, condition of rashes, tremors, vomit, urine, etc. By recording these items related to symptoms, the doctor can more accurately grasp the patient's condition. As a result, the reception unit can record items related to symptoms. Some or all of the above-described processing in the reception unit may be performed using AI or may be performed without using AI. For example, the reception unit can input the recorded video into AI and automatically extract parts related to symptoms.

The reception unit can record the condition of an injury. For example, the reception unit records the part of the body where the patient was injured, such as bleeding, fractures, bruises, etc. By recording these injury conditions, the doctor can more accurately grasp the patient's injury status. As a result, the reception unit can record the condition of an injury. Some or all of the above-described processing in the reception unit may be performed using AI or may be performed without using AI. For example, the reception unit can input the recorded video into AI and automatically extract the condition of the injury.

The reception unit can record the object of the injury. For example, the reception unit records the object that caused the patient's injury, such as the stairs that were fallen down, the ball that hit, or the insect that bit. By recording these objects of the injury, the doctor can more accurately grasp the cause of the patient's injury. As a result, the reception unit can record the object of the injury. Some or all of the above-described processing in the reception unit may be performed using AI or may be performed without using AI. For example, the reception unit can input the recorded video into AI and automatically extract the object of the injury.

The reading unit can read aloud the generated summary. For example, the reading unit reads aloud the generated summary by voice. The reading unit can read aloud the summary by voice using AI. As a result, the reading unit can read aloud the generated summary. Some or all of the above-described processing in the reading unit may be performed using AI or may be performed without using AI. For example, the reading unit can use speech synthesis technology to read aloud the generated summary.

The formatting unit can launch a medical examination format before the patient's arrival via communication. For example, the formatting unit automatically generates a medical examination format based on the generated summary and transmits it to the hospital. The formatting unit can automatically generate the medical examination format using AI. As a result, the formatting unit can launch a medical examination format before the patient's arrival via communication. Some or all of the above-described processing in the formatting unit may be performed using AI or may be performed without using AI. For example, the formatting unit can automatically generate a medical examination format based on the generated summary and transmit it to the hospital's electronic medical record system.

The reception unit can analyze the patient's past recording history and select an optimal recording method. For example, the reception unit analyzes the patterns of videos previously recorded by the patient and starts recording in a similar manner. The reception unit can also evaluate the quality of videos previously recorded by the patient and propose optimal camera settings. The reception unit can also analyze the content of videos previously recorded by the patient and present necessary information in advance. As a result, the reception unit can analyze the patient's past recording history and select an optimal recording method. Some or all of the above-described processing in the reception unit may be performed using AI or may be performed without using AI. For example, the reception unit can input the past recording history into AI and have the AI select the optimal recording method.

The reception unit can filter the recording content based on the patient's current health condition or symptoms at the time of recording. For example, if the patient has a high fever, the reception unit instructs to record the display of a thermometer. If the patient complains of a rash, the reception unit can also instruct to focus on recording the area of the rash. If the patient complains of difficulty breathing, the reception unit can also instruct to record the breathing condition. As a result, the reception unit can filter the recording content based on the patient's current health condition or symptoms. Some or all of the above-described processing in the reception unit may be performed using AI or may be performed without using AI. For example, the reception unit can input the patient's health condition or symptoms into AI and have the AI perform the filtering of the recording content.

The reception unit can prioritize recording content with high relevance by considering the patient's geographic location at the time of recording. For example, if the patient is in a high-altitude area, the reception unit instructs to record symptoms of altitude sickness. If the patient is at the seaside, the reception unit can also instruct to record symptoms of sunburn or heatstroke. If the patient is in an urban area, the reception unit can also instruct to record the situation of a traffic accident. As a result, the reception unit can prioritize recording content with high relevance by considering the patient's geographic location. Some or all of the above-described processing in the reception unit may be performed using AI or may be performed without using AI. For example, the reception unit can input the patient's geographic location into AI and have the AI select content with high relevance.

The reception unit can analyze the patient's social media activity at the time of recording and record relevant content. For example, the reception unit proposes recording content based on symptoms posted by the patient on social media. The reception unit can also determine the recording content by referring to health information previously posted by the patient. The reception unit can also adjust the recording content based on advice from medical professionals followed by the patient. As a result, the reception unit can analyze the patient's social media activity and record relevant content. Some or all of the above-described processing in the reception unit may be performed using AI or may be performed without using AI. For example, the reception unit can input the patient's social media activity into AI and have the AI select relevant content.

The analysis unit can apply different analysis algorithms based on the content of the video during analysis. For example, if the patient's symptoms are diverse, the analysis unit combines multiple analysis algorithms for analysis. If the patient's symptoms are concentrated in a specific area, the analysis unit can apply an analysis algorithm specialized for that area. If the patient's symptoms change over time, the analysis unit can apply a time-series analysis algorithm. As a result, the analysis unit can apply different analysis algorithms based on the content of the video. Some or all of the above-described processing in the analysis unit may be performed using AI or may be performed without using AI. For example, the analysis unit can input the content of the video into AI and have the AI select the optimal analysis algorithm.

The analysis unit can customize the analysis method according to the shooting environment or situation of the video during analysis. For example, if the video was shot in a dark environment, the analysis unit adjusts the brightness for analysis. If the video was shot in a noisy environment, the analysis unit can remove noise for analysis. If the video was shot in a highly dynamic environment, the analysis unit can correct for movement for analysis. As a result, the analysis unit can customize the analysis method according to the shooting environment or situation of the video. Some or all of the above-described processing in the analysis unit may be performed using AI or may be performed without using AI. For example, the analysis unit can input the shooting environment or situation of the video into AI and have the AI select the optimal analysis method.

The analysis unit can determine the priority of analysis based on the shooting time of the video during analysis. For example, the analysis unit prioritizes the analysis of the latest videos and provides results quickly. The analysis unit can postpone the analysis of older videos and focus on the latest information. If the shooting time is important, the analysis unit can prioritize the analysis of videos shot at a specific time. As a result, the analysis unit can determine the priority of analysis based on the shooting time of the video. Some or all of the above-described processing in the analysis unit may be performed using AI or may be performed without using AI. For example, the analysis unit can input the shooting time of the video into AI and have the AI determine the priority of analysis.

The analysis unit can refer to related literature of the video during analysis to improve the accuracy of the analysis. For example, the analysis unit refers to medical literature related to the content of the video to reinforce the analysis results. The analysis unit can refer to research papers related to the content of the video to improve the analysis algorithm. The analysis unit can refer to guidelines related to the content of the video to scrutinize the analysis results. As a result, the analysis unit can refer to related literature of the video to improve the accuracy of the analysis. Some or all of the above-described processing in the analysis unit may be performed using AI or may be performed without using AI. For example, the analysis unit can input the content of the video into AI and have the AI refer to related literature.

The generation unit can adjust the level of detail of the summary based on the importance of the video when generating the summary. For example, if the video contains important symptoms, the generation unit generates a detailed summary. If the video contains minor symptoms, the generation unit can generate a concise summary. If the video contains highly urgent symptoms, the generation unit can generate a summary that can be quickly understood. As a result, the generation unit can adjust the level of detail of the summary based on the importance of the video. Some or all of the above-described processing in the generation unit may be performed using AI or may be performed without using AI. For example, the generation unit can input the importance of the video into AI and have the AI adjust the level of detail of the summary.

The generation unit can apply different summarization algorithms according to the category of the video when generating the summary. For example, if the video is related to a disease, the generation unit applies a summarization algorithm specialized for diseases. If the video is related to an injury, the generation unit can apply a summarization algorithm specialized for injuries. If the video is related to other symptoms, the generation unit can apply a summarization algorithm according to the symptoms. As a result, the generation unit can apply different summarization algorithms according to the category of the video. Some or all of the above-described processing in the generation unit may be performed using AI or may be performed without using AI. For example, the generation unit can input the category of the video into AI and have the AI select the optimal summarization algorithm.

The generation unit can determine the priority of the summary based on the shooting time of the video when generating the summary. For example, the generation unit prioritizes the summarization of the latest videos and provides results quickly. The generation unit can postpone the summarization of older videos and focus on the latest information. If the shooting time is important, the generation unit can prioritize the summarization of videos shot at a specific time. As a result, the generation unit can determine the priority of the summary based on the shooting time of the video. Some or all of the above-described processing in the generation unit may be performed using AI or may be performed without using AI. For example, the generation unit can input the shooting time of the video into AI and have the AI determine the priority of the summary.

The generation unit can adjust the order of the summary based on the relevance of the video when generating the summary. For example, if the video contains important symptoms, the generation unit generates the summary first. If the video contains minor symptoms, the generation unit can generate the summary later. If the video contains highly urgent symptoms, the generation unit can generate the summary quickly. As a result, the generation unit can adjust the order of the summary based on the relevance of the video. Some or all of the above-described processing in the generation unit may be performed using AI or may be performed without using AI. For example, the generation unit can input the relevance of the video into AI and have the AI adjust the order of the summary.

The reading unit can apply different reading algorithms based on the content of the summary when reading aloud. For example, if the summary contains important symptoms, the reading unit reads aloud in detail. If the summary contains minor symptoms, the reading unit can read aloud concisely. If the summary contains highly urgent symptoms, the reading unit can read aloud quickly. As a result, the reading unit can apply different reading algorithms based on the content of the summary. Some or all of the above-described processing in the reading unit may be performed using AI or may be performed without using AI. For example, the reading unit can input the content of the summary into AI and have the AI select the optimal reading algorithm.

The reading unit can adjust the level of detail of reading aloud according to the importance of the summary when reading aloud. For example, if the summary contains important symptoms, the reading unit reads aloud in detail. If the summary contains minor symptoms, the reading unit can read aloud concisely. If the summary contains highly urgent symptoms, the reading unit can read aloud quickly. As a result, the reading unit can adjust the level of detail of reading aloud according to the importance of the summary. Some or all of the above-described processing in the reading unit may be performed using AI or may be performed without using AI. For example, the reading unit can input the importance of the summary into AI and have the AI adjust the level of detail of reading aloud.

The reading unit can determine the priority of reading aloud based on the shooting time of the summary when reading aloud. For example, the reading unit prioritizes reading aloud the latest summaries. The reading unit can postpone reading aloud older summaries and focus on the latest information. If the shooting time is important, the reading unit can prioritize reading aloud summaries shot at a specific time. As a result, the reading unit can determine the priority of reading aloud based on the shooting time of the summary. Some or all of the above-described processing in the reading unit may be performed using AI or may be performed without using AI. For example, the reading unit can input the shooting time of the summary into AI and have the AI determine the priority of reading aloud.

The reading unit can refer to related literature of the summary when reading aloud to improve the accuracy of reading aloud. For example, the reading unit refers to medical literature related to the content of the summary to reinforce the reading content. The reading unit can refer to research papers related to the content of the summary to improve the reading algorithm. The reading unit can refer to guidelines related to the content of the summary to scrutinize the reading content. As a result, the reading unit can refer to related literature of the summary to improve the accuracy of reading aloud. Some or all of the above-described processing in the reading unit may be performed using AI or may be performed without using AI. For example, the reading unit can input the content of the summary into AI and have the AI refer to related literature.

The formatting unit can apply different formatting algorithms based on the content of the summary when generating the medical examination format. For example, if the summary contains important symptoms, the formatting unit generates a detailed format. If the summary contains minor symptoms, the formatting unit can generate a concise format. If the summary contains highly urgent symptoms, the formatting unit can generate a format that can be quickly understood. As a result, the formatting unit can apply different formatting algorithms based on the content of the summary. Some or all of the above-described processing in the formatting unit may be performed using AI or may be performed without using AI. For example, the formatting unit can input the content of the summary into AI and have the AI select the optimal formatting algorithm.

The formatting unit can adjust the level of detail of the format according to the importance of the summary when generating the medical examination format. For example, if the summary contains important symptoms, the formatting unit generates a detailed format. If the summary contains minor symptoms, the formatting unit can generate a concise format. If the summary contains highly urgent symptoms, the formatting unit can generate a format that can be quickly understood. As a result, the formatting unit can adjust the level of detail of the format according to the importance of the summary. Some or all of the above-described processing in the formatting unit may be performed using AI or may be performed without using AI. For example, the formatting unit can input the importance of the summary into AI and have the AI adjust the level of detail of the format.

The formatting unit can determine the priority of the format based on the shooting time of the summary when generating the medical examination format. For example, the formatting unit prioritizes reflecting the latest summaries in the format. The formatting unit can postpone reflecting older summaries and focus on the latest information. If the shooting time is important, the formatting unit can prioritize reflecting summaries shot at a specific time in the format. As a result, the formatting unit can determine the priority of the format based on the shooting time of the summary. Some or all of the above-described processing in the formatting unit may be performed using AI or may be performed without using AI. For example, the formatting unit can input the shooting time of the summary into AI and have the AI determine the priority of the format.

The formatting unit can refer to related literature of the summary when generating the medical examination format to improve the accuracy of the format. For example, the formatting unit refers to medical literature related to the content of the summary to reinforce the format content. The formatting unit can refer to research papers related to the content of the summary to improve the formatting algorithm. The formatting unit can refer to guidelines related to the content of the summary to scrutinize the format content. As a result, the formatting unit can refer to related literature of the summary to improve the accuracy of the format. Some or all of the above-described processing in the formatting unit may be performed using AI or may be performed without using AI. For example, the formatting unit can input the content of the summary into AI and have the AI refer to related literature.

The system according to the embodiment is not limited to the above examples and can be variously modified as described below, for example.

The reception unit can analyze the patient's past recording history and select an optimal recording method. For example, the reception unit analyzes the patterns of videos previously recorded by the patient and starts recording in a similar manner. The reception unit can also evaluate the quality of videos previously recorded by the patient and propose optimal camera settings. The reception unit can also analyze the content of videos previously recorded by the patient and present necessary information in advance. As a result, the reception unit can analyze the patient's past recording history and select an optimal recording method. Some or all of the above-described processing in the reception unit may be performed using AI or may be performed without using AI. For example, the reception unit can input the past recording history into AI and have the AI select the optimal recording method.

The reception unit can filter the recording content based on the patient's current health condition or symptoms at the time of recording. For example, if the patient has a high fever, the reception unit instructs to record the display of a thermometer. If the patient complains of a rash, the reception unit can also instruct to focus on recording the area of the rash. If the patient complains of difficulty breathing, the reception unit can also instruct to record the breathing condition. As a result, the reception unit can filter the recording content based on the patient's current health condition or symptoms. Some or all of the above-described processing in the reception unit may be performed using AI or may be performed without using AI. For example, the reception unit can input the patient's health condition or symptoms into AI and have the AI perform the filtering of the recording content.

The analysis unit can apply different analysis algorithms based on the content of the video during analysis. For example, if the patient's symptoms are diverse, the analysis unit combines multiple analysis algorithms for analysis. If the patient's symptoms are concentrated in a specific area, the analysis unit can apply an analysis algorithm specialized for that area. If the patient's symptoms change over time, the analysis unit can apply a time-series analysis algorithm. As a result, the analysis unit can apply different analysis algorithms based on the content of the video. Some or all of the above-described processing in the analysis unit may be performed using AI or may be performed without using AI. For example, the analysis unit can input the content of the video into AI and have the AI select the optimal analysis algorithm.

The generation unit can adjust the level of detail of the summary based on the importance of the video when generating the summary. For example, if the video contains important symptoms, the generation unit generates a detailed summary. If the video contains minor symptoms, the generation unit can generate a concise summary. If the video contains highly urgent symptoms, the generation unit can generate a summary that can be quickly understood. As a result, the generation unit can adjust the level of detail of the summary based on the importance of the video. Some or all of the above-described processing in the generation unit may be performed using AI or may be performed without using AI. For example, the generation unit can input the importance of the video into AI and have the AI adjust the level of detail of the summary.

The reading unit can apply different reading algorithms based on the content of the summary when reading aloud. For example, if the summary contains important symptoms, the reading unit reads aloud in detail. If the summary contains minor symptoms, the reading unit can read aloud concisely. If the summary contains highly urgent symptoms, the reading unit can read aloud quickly. As a result, the reading unit can apply different reading algorithms based on the content of the summary. Some or all of the above-described processing in the reading unit may be performed using AI or may be performed without using AI. For example, the reading unit can input the content of the summary into AI and have the AI select the optimal reading algorithm.

The following is a brief description of the processing flow of Example 1 of the Embodiment.
Step 1: The reception unit records videos recorded by the patient or a related person. The videos recorded by the patient or a related person include, for example, videos in which the condition of the affected area or symptoms are described. The reception unit uses a smartphone or tablet to record the video and has a function to upload the recorded video to the cloud.
Step 2: The analysis unit analyzes the videos recorded by the reception unit. The analysis unit analyzes the content of the video and extracts symptoms and the condition of the affected area. The analysis unit can analyze the content of the video using AI.
Step 3: The generation unit generates a summary based on the videos analyzed by the analysis unit. The generation unit generates the summary using generative AI and summarizes the content of the video, converting it into a format that is easy for the doctor to understand.
Step 4: The reading unit reads aloud the summary generated by the generation unit. The reading unit can read aloud the summary by voice using AI.
Step 5: The formatting unit launches a medical examination format based on the summary generated by the generation unit. The formatting unit can automatically generate the medical examination format using AI.

### Example 2 of Embodiment

The AI system according to the embodiment of the present invention is a system that summarizes videos recorded by a patient or a related person into content that is easy for a doctor to understand and acts as a bridge. In this AI system, the patient or a related person records the affected area while talking about symptoms. For example, symptoms such as “dull pain,” “nausea,” or “faintness” are recorded. In addition, items related to symptoms are recorded, such as complexion, condition of rashes, tremors, vomit, urine, etc. Next, in the case of an injury, the condition of the affected area and the object of the injury are recorded, such as the stairs that were fallen down, the ball that hit, or the insect that bit. These videos are analyzed by AI before arriving at the hospital and summarized for the doctor. Since the patient may not be able to speak after arrival, a summary reading function is also provided. Furthermore, as a second phase, it is also possible to have the medical examination format launched on the hospital side before the patient's arrival via communication. This AI system is particularly targeted at foreigners who are not proficient in Japanese, inbound tourists, young people who cannot explain their symptoms, elderly people with cognitive decline, and patients with severe symptoms who cannot speak. As a result, it is no longer necessary to explain verbally one-on-one to the doctor, and accurate information can be provided even if the memory is vague. In addition, the doctor can easily grasp the situation at the time of occurrence and eliminate language barriers and differences in nuance. This system bridges the person who knows the situation at the time of occurrence (the patient) and the person who provides treatment (the doctor), thereby eliminating recognition discrepancies. It is also possible to fill the time gap between occurrence and treatment. Furthermore, generative AI technologies such as summarization, translation, video analysis, emotion analysis, and language analysis are utilized. As a result, the AI system can summarize the patient's video into content that is easy for the doctor to understand and act as a bridge.

The AI system according to the embodiment includes a reception unit, an analysis unit, a generation unit, a reading unit, and a formatting unit. The reception unit records videos recorded by the patient or a related person. The videos recorded by the patient or a related person include, for example, videos in which the condition of the affected area or symptoms are described. The reception unit can record videos using, for example, a smartphone or tablet. The reception unit also has a function to upload the recorded videos to the cloud. The analysis unit analyzes the videos recorded by the reception unit. The analysis unit analyzes, for example, the content of the videos and extracts symptoms and the condition of the affected area. The analysis unit can analyze the content of the videos using AI. The generation unit generates a summary based on the videos analyzed by the analysis unit. The generation unit generates the summary using generative AI. The generation unit, for example, summarizes the content of the videos and converts it into a format that is easy for the doctor to understand. The reading unit reads aloud the summary generated by the generation unit. The reading unit can read aloud the summary using AI. The formatting unit launches a medical examination format based on the summary generated by the generation unit. The formatting unit can automatically generate the medical examination format using AI. Thus, the AI system according to the embodiment can summarize the patient's video into content that is easy for the doctor to understand and act as a bridge.

The reception unit records videos recorded by the patient or a related person. The videos recorded by the patient or a related person include, for example, videos in which the condition of the affected area or symptoms are described. The reception unit can record videos using, for example, a smartphone or tablet. Specifically, the patient uses the camera of a smartphone to shoot detailed images of the affected area and records a video in which the symptoms, degree of pain, and onset time are verbally explained. As a result, even if the doctor cannot examine the patient directly, detailed information can be provided. The reception unit also has a function to upload the recorded videos to the cloud. Uploading to the cloud is performed in an encrypted manner to ensure security and protect the patient's privacy. Furthermore, the reception unit also has a function to notify when the video upload is complete, allowing the patient or related person to check the transmission status of the video. Thus, the reception unit can efficiently and securely collect the patient's videos and quickly move to the next analysis step.

The analysis unit analyzes the videos recorded by the reception unit. The analysis unit analyzes, for example, the content of the videos and extracts symptoms and the condition of the affected area. The analysis unit can analyze the content of the videos using AI. Specifically, the AI uses image recognition technology for each frame of the video to analyze the condition of the affected area and detect changes in color, degree of swelling, presence or absence of bleeding, etc. In addition, speech recognition technology is used to convert the patient's explanation into text and extract information such as details of symptoms, onset time, and degree of pain. Furthermore, natural language processing technology is used to analyze the extracted text data and identify important keywords and phrases. As a result, the analysis unit can integrate visual and audio information obtained from the video and grasp the patient's symptoms and the condition of the affected area in detail. The analysis unit provides important data for the doctor to make a diagnosis based on this information.

The generation unit generates a summary based on the videos analyzed by the analysis unit. The generation unit generates the summary using generative AI. Specifically, the generative AI generates a summary in a format that is easy for the doctor to understand based on the text data and image data provided by the analysis unit. For example, the generative AI concisely summarizes the patient's symptoms and the condition of the affected area and emphasizes important points. In addition, the generative AI adjusts the summary to preferentially include information necessary for the doctor to make a diagnosis. As a result, the generation unit provides support for the doctor to quickly grasp the patient's condition and make an appropriate diagnosis. Furthermore, the generation unit can refer to past diagnostic data and medical literature to improve the accuracy of the summary and generate the optimal summary. Thus, the generation unit can always provide high-quality summaries reflecting the latest medical knowledge.

The reading unit reads aloud the summary generated by the generation unit. The reading unit can read aloud the summary using AI. Specifically, the reading unit uses speech synthesis technology to read aloud the generated summary in a natural voice. The speech synthesis technology converts text data into audio data and can read aloud with natural intonation and pronunciation. As a result, the doctor can not only visually check the summary but also listen to it by voice, thereby improving the efficiency of diagnosis. Furthermore, the reading unit has functions to adjust the reading speed and volume, allowing customization according to the doctor's preferences. Thus, the reading unit supports the doctor in obtaining information in the most efficient way. In addition, the reading unit can also support multiple languages and can read aloud the summary in different languages, making it useful in international medical settings.

The formatting unit launches a medical examination format based on the summary generated by the generation unit. The formatting unit can automatically generate the medical examination format using AI. Specifically, the formatting unit organizes the information necessary for the examination based on the generated summary and converts it into a standardized format. For example, the formatting unit automatically creates a medical examination format including the patient's basic information, details of symptoms, condition of the affected area, past medical history, etc. As a result, the doctor can save the trouble of manually creating the medical examination format and concentrate on diagnosis. Furthermore, the formatting unit can link the medical examination format with the electronic medical record system and quickly record the examination results. This improves the efficiency of medical examinations and reduces the workload in medical settings. In addition, the formatting unit can also customize the medical examination format and flexibly respond to the doctor's needs. Thus, the formatting unit provides support for the doctor to perform optimal examinations and can improve the treatment effect for the patient.

The reception unit can record a video in which a patient or a related person talks about symptoms while recording the affected area. For example, the reception unit records the patient talking about symptoms such as “dull pain,” “nausea,” or “faintness” while recording the affected area. In addition, the reception unit can also record items related to symptoms, such as complexion, condition of rashes, tremors, vomit, urine, etc. As a result, the reception unit can record a video in which a patient or a related person talks about symptoms while recording the affected area. Some or all of the above-described processing in the reception unit may be performed using AI or may be performed without using AI. For example, the reception unit can use speech recognition technology to convert the content spoken by the patient into text and store it together with the recorded content.

The reception unit can record items related to symptoms. For example, the reception unit records the patient's complexion, condition of rashes, tremors, vomit, urine, etc. By recording these items related to symptoms, the doctor can more accurately grasp the patient's condition. As a result, the reception unit can record items related to symptoms. Some or all of the above-described processing in the reception unit may be performed using AI or may be performed without using AI. For example, the reception unit can input the recorded video into AI and automatically extract parts related to symptoms.

The reception unit can record the condition of an injury. For example, the reception unit records the part of the body where the patient was injured, such as bleeding, fractures, bruises, etc. By recording these injury conditions, the doctor can more accurately grasp the patient's injury status. As a result, the reception unit can record the condition of an injury. Some or all of the above-described processing in the reception unit may be performed using AI or may be performed without using AI. For example, the reception unit can input the recorded video into AI and automatically extract the condition of the injury.

The reception unit can record the object of the injury. For example, the reception unit records the object that caused the patient's injury, such as the stairs that were fallen down, the ball that hit, or the insect that bit. By recording these objects of the injury, the doctor can more accurately grasp the cause of the patient's injury. As a result, the reception unit can record the object of the injury. Some or all of the above-described processing in the reception unit may be performed using AI or may be performed without using AI. For example, the reception unit can input the recorded video into AI and automatically extract the object of the injury.

The reading unit can read aloud the generated summary. For example, the reading unit reads aloud the generated summary by voice. The reading unit can read aloud the summary by voice using AI. As a result, the reading unit can read aloud the generated summary. Some or all of the above-described processing in the reading unit may be performed using AI or may be performed without using AI. For example, the reading unit can use speech synthesis technology to read aloud the generated summary.

The formatting unit can launch a medical examination format before the patient's arrival via communication. For example, the formatting unit automatically generates a medical examination format based on the generated summary and transmits it to the hospital. The formatting unit can automatically generate the medical examination format using AI. As a result, the formatting unit can launch a medical examination format before the patient's arrival via communication. Some or all of the above-described processing in the formatting unit may be performed using AI or may be performed without using AI. For example, the formatting unit can automatically generate a medical examination format based on the generated summary and transmit it to the hospital's electronic medical record system.

The reception unit can estimate the patient's emotion and adjust the timing for starting the recording based on the estimated emotion. For example, if the patient is nervous, the reception unit waits until the patient relaxes before starting the recording. If the patient is anxious, the reception unit can start recording immediately and edit later. If the patient is calm, the reception unit can start recording before requesting a detailed explanation. As a result, the reception unit can adjust the timing for starting the recording based on the patient's emotion. Emotion estimation is realized using an emotion engine or a generative AI with an emotion estimation function. The generative AI may be a text generation AI (e.g., LLM) or a multimodal generative AI, but is not limited to such examples. Some or all of the above-described processing in the reception unit may be performed using AI or may be performed without using AI. For example, the reception unit can input facial expression data of the patient captured by a camera into the generative AI and have the generative AI perform the emotion estimation of the patient.

The reception unit can analyze the patient's past recording history and select an optimal recording method. For example, the reception unit analyzes the patterns of videos previously recorded by the patient and starts recording in a similar manner. The reception unit can also evaluate the quality of videos previously recorded by the patient and propose optimal camera settings. The reception unit can also analyze the content of videos previously recorded by the patient and present necessary information in advance. As a result, the reception unit can analyze the patient's past recording history and select an optimal recording method. Some or all of the above-described processing in the reception unit may be performed using AI or may be performed without using AI. For example, the reception unit can input the past recording history into AI and have the AI select the optimal recording method.

The reception unit can filter the recording content based on the patient's current health condition or symptoms at the time of recording. For example, if the patient has a high fever, the reception unit instructs to record the display of a thermometer. If the patient complains of a rash, the reception unit can also instruct to focus on recording the area of the rash. If the patient complains of difficulty breathing, the reception unit can also instruct to record the breathing condition. As a result, the reception unit can filter the recording content based on the patient's current health condition or symptoms. Some or all of the above-described processing in the reception unit may be performed using AI or may be performed without using AI. For example, the reception unit can input the patient's health condition or symptoms into AI and have the AI perform the filtering of the recording content.

The reception unit can estimate the patient's emotion and determine the priority of the content to be recorded based on the estimated emotion. For example, if the patient feels anxious, the reception unit first records a reassuring message. If the patient complains of pain, the reception unit can prioritize recording the area of pain. If the patient is confused, the reception unit can start recording with a simple explanation. As a result, the reception unit can determine the priority of the content to be recorded based on the patient's emotion. Emotion estimation is realized using an emotion engine or a generative AI with an emotion estimation function. The generative AI may be a text generation AI (e.g., LLM) or a multimodal generative AI, but is not limited to such examples. Some or all of the above-described processing in the reception unit may be performed using AI or may be performed without using AI. For example, the reception unit can input facial expression data of the patient captured by a camera into the generative AI and have the generative AI perform the emotion estimation of the patient.

The reception unit can prioritize recording content with high relevance by considering the patient's geographic location at the time of recording. For example, if the patient is in a high-altitude area, the reception unit instructs to record symptoms of altitude sickness. If the patient is at the seaside, the reception unit can also instruct to record symptoms of sunburn or heatstroke. If the patient is in an urban area, the reception unit can also instruct to record the situation of a traffic accident. As a result, the reception unit can prioritize recording content with high relevance by considering the patient's geographic location. Some or all of the above-described processing in the reception unit may be performed using AI or may be performed without using AI. For example, the reception unit can input the patient's geographic location into AI and have the AI select content with high relevance.

The reception unit can analyze the patient's social media activity at the time of recording and record relevant content. For example, the reception unit proposes recording content based on symptoms posted by the patient on social media. The reception unit can also determine the recording content by referring to health information previously posted by the patient. The reception unit can also adjust the recording content based on advice from medical professionals followed by the patient. As a result, the reception unit can analyze the patient's social media activity and record relevant content. Some or all of the above-described processing in the reception unit may be performed using AI or may be performed without using AI. For example, the reception unit can input the patient's social media activity into AI and have the AI select relevant content.

The analysis unit can estimate the patient's emotion and adjust the analysis accuracy based on the estimated emotion. For example, if the patient is nervous, the analysis unit increases the accuracy to provide detailed information. If the patient is relaxed, the analysis unit can maintain the accuracy at a normal level. If the patient is anxious, the analysis unit can perform rapid analysis and provide results quickly. As a result, the analysis unit can adjust the analysis accuracy based on the patient's emotion. Emotion estimation is realized using an emotion engine or a generative AI with an emotion estimation function. The generative AI may be a text generation AI (e.g., LLM) or a multimodal generative AI, but is not limited to such examples. Some or all of the above-described processing in the analysis unit may be performed using AI or may be performed without using AI. For example, the analysis unit can input facial expression data of the patient captured by a camera into the generative AI and have the generative AI perform the emotion estimation of the patient.

The analysis unit can apply different analysis algorithms based on the content of the video during analysis. For example, if the patient's symptoms are diverse, the analysis unit combines multiple analysis algorithms for analysis. If the patient's symptoms are concentrated in a specific area, the analysis unit can apply an analysis algorithm specialized for that area. If the patient's symptoms change over time, the analysis unit can apply a time-series analysis algorithm. As a result, the analysis unit can apply different analysis algorithms based on the content of the video. Some or all of the above-described processing in the analysis unit may be performed using AI or may be performed without using AI. For example, the analysis unit can input the content of the video into AI and have the AI select the optimal analysis algorithm.

The analysis unit can customize the analysis method according to the shooting environment or situation of the video during analysis. For example, if the video was shot in a dark environment, the analysis unit adjusts the brightness for analysis. If the video was shot in a noisy environment, the analysis unit can remove noise for analysis. If the video was shot in a highly dynamic environment, the analysis unit can correct for movement for analysis. As a result, the analysis unit can customize the analysis method according to the shooting environment or situation of the video. Some or all of the above-described processing in the analysis unit may be performed using AI or may be performed without using AI. For example, the analysis unit can input the shooting environment or situation of the video into AI and have the AI select the optimal analysis method.

The analysis unit can estimate the patient's emotion and adjust the display method of the analysis result based on the estimated emotion. For example, if the patient is nervous, the analysis unit provides a simple and highly visible display method. If the patient is relaxed, the analysis unit can provide a display method that includes detailed information. If the patient is anxious, the analysis unit can provide a display method that focuses on key points. As a result, the analysis unit can adjust the display method of the analysis result based on the patient's emotion. Emotion estimation is realized using an emotion engine or a generative AI with an emotion estimation function. The generative AI may be a text generation AI (e.g., LLM) or a multimodal generative AI, but is not limited to such examples. Some or all of the above-described processing in the analysis unit may be performed using AI or may be performed without using AI. For example, the analysis unit can input facial expression data of the patient captured by a camera into the generative AI and have the generative AI perform the emotion estimation of the patient.

The analysis unit can determine the priority of analysis based on the shooting time of the video during analysis. For example, the analysis unit prioritizes the analysis of the latest videos and provides results quickly. The analysis unit can postpone the analysis of older videos and focus on the latest information. If the shooting time is important, the analysis unit can prioritize the analysis of videos shot at a specific time. As a result, the analysis unit can determine the priority of analysis based on the shooting time of the video. Some or all of the above-described processing in the analysis unit may be performed using AI or may be performed without using AI. For example, the analysis unit can input the shooting time of the video into AI and have the AI determine the priority of analysis.

The analysis unit can refer to related literature of the video during analysis to improve the accuracy of the analysis. For example, the analysis unit refers to medical literature related to the content of the video to reinforce the analysis results. The analysis unit can refer to research papers related to the content of the video to improve the analysis algorithm. The analysis unit can refer to guidelines related to the content of the video to scrutinize the analysis results. As a result, the analysis unit can refer to related literature of the video to improve the accuracy of the analysis. Some or all of the above-described processing in the analysis unit may be performed using AI or may be performed without using AI. For example, the analysis unit can input the content of the video into AI and have the AI refer to related literature.

The generation unit can estimate the patient's emotion and adjust the expression method of the summary based on the estimated emotion. For example, if the patient is nervous, the generation unit generates a concise and easy-to-understand summary. If the patient is relaxed, the generation unit can generate a summary that includes detailed information. If the patient is anxious, the generation unit can generate a summary that can be quickly understood. As a result, the generation unit can adjust the expression method of the summary based on the patient's emotion. Emotion estimation is realized using an emotion engine or a generative AI with an emotion estimation function. The generative AI may be a text generation AI (e.g., LLM) or a multimodal generative AI, but is not limited to such examples. Some or all of the above-described processing in the generation unit may be performed using AI or may be performed without using AI. For example, the generation unit can input facial expression data of the patient captured by a camera into the generative AI and have the generative AI perform the emotion estimation of the patient.

The generation unit can adjust the level of detail of the summary based on the importance of the video when generating the summary. For example, if the video contains important symptoms, the generation unit generates a detailed summary. If the video contains minor symptoms, the generation unit can generate a concise summary. If the video contains highly urgent symptoms, the generation unit can generate a summary that can be quickly understood. As a result, the generation unit can adjust the level of detail of the summary based on the importance of the video. Some or all of the above-described processing in the generation unit may be performed using AI or may be performed without using AI. For example, the generation unit can input the importance of the video into AI and have the AI adjust the level of detail of the summary.

The generation unit can apply different summarization algorithms according to the category of the video when generating the summary. For example, if the video is related to a disease, the generation unit applies a summarization algorithm specialized for diseases. If the video is related to an injury, the generation unit can apply a summarization algorithm specialized for injuries. If the video is related to other symptoms, the generation unit can apply a summarization algorithm according to the symptoms. As a result, the generation unit can apply different summarization algorithms according to the category of the video. Some or all of the above-described processing in the generation unit may be performed using AI or may be performed without using AI. For example, the generation unit can input the category of the video into AI and have the AI select the optimal summarization algorithm.

The generation unit can estimate the patient's emotion and adjust the length of the summary based on the estimated emotion. For example, if the patient is nervous, the generation unit generates a short and to-the-point summary. If the patient is relaxed, the generation unit can generate a longer summary that includes detailed explanations. If the patient is anxious, the generation unit can generate a short summary that can be quickly understood. As a result, the generation unit can adjust the length of the summary based on the patient's emotion. Emotion estimation is realized using an emotion engine or a generative AI with an emotion estimation function. The generative AI may be a text generation AI (e.g., LLM) or a multimodal generative AI, but is not limited to such examples. Some or all of the above-described processing in the generation unit may be performed using AI or may be performed without using AI. For example, the generation unit can input facial expression data of the patient captured by a camera into the generative AI and have the generative AI perform the emotion estimation of the patient.

The generation unit can determine the priority of the summary based on the shooting time of the video when generating the summary. For example, the generation unit prioritizes the summarization of the latest videos and provides results quickly. The generation unit can postpone the summarization of older videos and focus on the latest information. If the shooting time is important, the generation unit can prioritize the summarization of videos shot at a specific time. As a result, the generation unit can determine the priority of the summary based on the shooting time of the video. Some or all of the above-described processing in the generation unit may be performed using AI or may be performed without using AI. For example, the generation unit can input the shooting time of the video into AI and have the AI determine the priority of the summary.

The generation unit can adjust the order of the summary based on the relevance of the video when generating the summary. For example, if the video contains important symptoms, the generation unit generates the summary first. If the video contains minor symptoms, the generation unit can generate the summary later. If the video contains highly urgent symptoms, the generation unit can generate the summary quickly. As a result, the generation unit can adjust the order of the summary based on the relevance of the video. Some or all of the above-described processing in the generation unit may be performed using AI or may be performed without using AI. For example, the generation unit can input the relevance of the video into AI and have the AI adjust the order of the summary.

The reading unit can estimate the patient's emotion and adjust the tone and speed of reading aloud based on the estimated emotion. For example, if the patient is nervous, the reading unit reads aloud slowly in a calm tone. If the patient is relaxed, the reading unit can read aloud in a bright tone. If the patient is anxious, the reading unit can perform quick and concise reading. As a result, the reading unit can adjust the tone and speed of reading aloud based on the patient's emotion. Emotion estimation is realized using an emotion engine or a generative AI with an emotion estimation function. The generative AI may be a text generation AI (e.g., LLM) or a multimodal generative AI, but is not limited to such examples. Some or all of the above-described processing in the reading unit may be performed using AI or may be performed without using AI. For example, the reading unit can input facial expression data of the patient captured by a camera into the generative AI and have the generative AI perform the emotion estimation of the patient.

The reading unit can apply different reading algorithms based on the content of the summary when reading aloud. For example, if the summary contains important symptoms, the reading unit reads aloud in detail. If the summary contains minor symptoms, the reading unit can read aloud concisely. If the summary contains highly urgent symptoms, the reading unit can read aloud quickly. As a result, the reading unit can apply different reading algorithms based on the content of the summary. Some or all of the above-described processing in the reading unit may be performed using AI or may be performed without using AI. For example, the reading unit can input the content of the summary into AI and have the AI select the optimal reading algorithm.

The reading unit can adjust the level of detail of reading aloud according to the importance of the summary when reading aloud. For example, if the summary contains important symptoms, the reading unit reads aloud in detail. If the summary contains minor symptoms, the reading unit can read aloud concisely. If the summary contains highly urgent symptoms, the reading unit can read aloud quickly. As a result, the reading unit can adjust the level of detail of reading aloud according to the importance of the summary. Some or all of the above-described processing in the reading unit may be performed using AI or may be performed without using AI. For example, the reading unit can input the importance of the summary into AI and have the AI adjust the level of detail of reading aloud.

The reading unit can estimate the patient's emotion and adjust the order of reading aloud based on the estimated emotion. For example, if the patient is nervous, the reading unit reads aloud important information first. If the patient is relaxed, the reading unit can postpone detailed information and read it aloud later. If the patient is anxious, the reading unit can read aloud information that can be quickly understood first. As a result, the reading unit can adjust the order of reading aloud based on the patient's emotion. Emotion estimation is realized using an emotion engine or a generative AI with an emotion estimation function. The generative AI may be a text generation AI (e.g., LLM) or a multimodal generative AI, but is not limited to such examples. Some or all of the above-described processing in the reading unit may be performed using AI or may be performed without using AI. For example, the reading unit can input facial expression data of the patient captured by a camera into the generative AI and have the generative AI perform the emotion estimation of the patient.

The reading unit can determine the priority of reading aloud based on the shooting time of the summary when reading aloud. For example, the reading unit prioritizes reading aloud the latest summaries. The reading unit can postpone reading aloud older summaries and focus on the latest information. If the shooting time is important, the reading unit can prioritize reading aloud summaries shot at a specific time. As a result, the reading unit can determine the priority of reading aloud based on the shooting time of the summary. Some or all of the above-described processing in the reading unit may be performed using AI or may be performed without using AI. For example, the reading unit can input the shooting time of the summary into AI and have the AI determine the priority of reading aloud.

The reading unit can refer to related literature of the summary when reading aloud to improve the accuracy of reading aloud. For example, the reading unit refers to medical literature related to the content of the summary to reinforce the reading content. The reading unit can refer to research papers related to the content of the summary to improve the reading algorithm. The reading unit can refer to guidelines related to the content of the summary to scrutinize the reading content. As a result, the reading unit can refer to related literature of the summary to improve the accuracy of reading aloud. Some or all of the above-described processing in the reading unit may be performed using AI or may be performed without using AI. For example, the reading unit can input the content of the summary into AI and have the AI refer to related literature.

The formatting unit can estimate the patient's emotion and adjust the display method of the medical examination format based on the estimated emotion. For example, if the patient is nervous, the formatting unit provides a simple and highly visible format. If the patient is relaxed, the formatting unit can provide a format that includes detailed information. If the patient is anxious, the formatting unit can provide a format that focuses on key points. As a result, the formatting unit can adjust the display method of the medical examination format based on the patient's emotion. Emotion estimation is realized using an emotion engine or a generative AI with an emotion estimation function. The generative AI may be a text generation AI (e.g., LLM) or a multimodal generative AI, but is not limited to such examples. Some or all of the above-described processing in the formatting unit may be performed using AI or may be performed without using AI. For example, the formatting unit can input facial expression data of the patient captured by a camera into the generative AI and have the generative AI perform the emotion estimation of the patient.

The formatting unit can apply different formatting algorithms based on the content of the summary when generating the medical examination format. For example, if the summary contains important symptoms, the formatting unit generates a detailed format. If the summary contains minor symptoms, the formatting unit can generate a concise format. If the summary contains highly urgent symptoms, the formatting unit can generate a format that can be quickly understood. As a result, the formatting unit can apply different formatting algorithms based on the content of the summary. Some or all of the above-described processing in the formatting unit may be performed using AI or may be performed without using AI. For example, the formatting unit can input the content of the summary into AI and have the AI select the optimal formatting algorithm.

The formatting unit can adjust the level of detail of the format according to the importance of the summary when generating the medical examination format. For example, if the summary contains important symptoms, the formatting unit generates a detailed format. If the summary contains minor symptoms, the formatting unit can generate a concise format. If the summary contains highly urgent symptoms, the formatting unit can generate a format that can be quickly understood. As a result, the formatting unit can adjust the level of detail of the format according to the importance of the summary. Some or all of the above-described processing in the formatting unit may be performed using AI or may be performed without using AI. For example, the formatting unit can input the importance of the summary into AI and have the AI adjust the level of detail of the format.

The formatting unit can estimate the patient's emotion and adjust the order of the medical examination format based on the estimated emotion. For example, if the patient is nervous, the formatting unit displays important information first. If the patient is relaxed, the formatting unit can postpone detailed information and display it later. If the patient is anxious, the formatting unit can display information that can be quickly understood first. As a result, the formatting unit can adjust the order of the medical examination format based on the patient's emotion. Emotion estimation is realized using an emotion engine or a generative AI with an emotion estimation function. The generative AI may be a text generation AI (e.g., LLM) or a multimodal generative AI, but is not limited to such examples. Some or all of the above-described processing in the formatting unit may be performed using AI or may be performed without using AI. For example, the formatting unit can input facial expression data of the patient captured by a camera into the generative AI and have the generative AI perform the emotion estimation of the patient.

The formatting unit can determine the priority of the format based on the shooting time of the summary when generating the medical examination format. For example, the formatting unit prioritizes reflecting the latest summaries in the format. The formatting unit can postpone reflecting older summaries and focus on the latest information. If the shooting time is important, the formatting unit can prioritize reflecting summaries shot at a specific time in the format. As a result, the formatting unit can determine the priority of the format based on the shooting time of the summary. Some or all of the above-described processing in the formatting unit may be performed using AI or may be performed without using AI. For example, the formatting unit can input the shooting time of the summary into AI and have the AI determine the priority of the format.

The formatting unit can refer to related literature of the summary when generating the medical examination format to improve the accuracy of the format. For example, the formatting unit refers to medical literature related to the content of the summary to reinforce the format content. The formatting unit can refer to research papers related to the content of the summary to improve the formatting algorithm. The formatting unit can refer to guidelines related to the content of the summary to scrutinize the format content. As a result, the formatting unit can refer to related literature of the summary to improve the accuracy of the format. Some or all of the above-described processing in the formatting unit may be performed using AI or may be performed without using AI. For example, the formatting unit can input the content of the summary into AI and have the AI refer to related literature.

The system according to the embodiment is not limited to the above examples and can be variously modified as described below, for example.

The reception unit can estimate the patient's emotion and adjust the timing for starting the recording based on the estimated emotion. For example, if the patient is nervous, the reception unit waits until the patient relaxes before starting the recording. If the patient is anxious, the reception unit can start recording immediately and edit later. If the patient is calm, the reception unit can start recording before requesting a detailed explanation. As a result, the reception unit can adjust the timing for starting the recording based on the patient's emotion. Emotion estimation is realized using an emotion engine or a generative AI with an emotion estimation function. The generative AI may be a text generation AI (e.g., LLM) or a multimodal generative AI, but is not limited to such examples. Some or all of the above-described processing in the reception unit may be performed using AI or may be performed without using AI. For example, the reception unit can input facial expression data of the patient captured by a camera into the generative AI and have the generative AI perform the emotion estimation of the patient.

The analysis unit can estimate the patient's emotion and adjust the analysis accuracy based on the estimated emotion. For example, if the patient is nervous, the analysis unit increases the accuracy to provide detailed information. If the patient is relaxed, the analysis unit can maintain the accuracy at a normal level. If the patient is anxious, the analysis unit can perform rapid analysis and provide results quickly. As a result, the analysis unit can adjust the analysis accuracy based on the patient's emotion. Emotion estimation is realized using an emotion engine or a generative AI with an emotion estimation function. The generative AI may be a text generation AI (e.g., LLM) or a multimodal generative AI, but is not limited to such examples. Some or all of the above-described processing in the analysis unit may be performed using AI or may be performed without using AI. For example, the analysis unit can input facial expression data of the patient captured by a camera into the generative AI and have the generative AI perform the emotion estimation of the patient.

The generation unit can estimate the patient's emotion and adjust the expression method of the summary based on the estimated emotion. For example, if the patient is nervous, the generation unit generates a concise and easy-to-understand summary. If the patient is relaxed, the generation unit can generate a summary that includes detailed information. If the patient is anxious, the generation unit can generate a summary that can be quickly understood. As a result, the generation unit can adjust the expression method of the summary based on the patient's emotion. Emotion estimation is realized using an emotion engine or a generative AI with an emotion estimation function. The generative AI may be a text generation AI (e.g., LLM) or a multimodal generative AI, but is not limited to such examples. Some or all of the above-described processing in the generation unit may be performed using AI or may be performed without using AI. For example, the generation unit can input facial expression data of the patient captured by a camera into the generative AI and have the generative AI perform the emotion estimation of the patient.

The reading unit can estimate the patient's emotion and adjust the tone and speed of reading aloud based on the estimated emotion. For example, if the patient is nervous, the reading unit reads aloud slowly in a calm tone. If the patient is relaxed, the reading unit can read aloud in a bright tone. If the patient is anxious, the reading unit can perform quick and concise reading. As a result, the reading unit can adjust the tone and speed of reading aloud based on the patient's emotion. Emotion estimation is realized using an emotion engine or a generative AI with an emotion estimation function. The generative AI may be a text generation AI (e.g., LLM) or a multimodal generative AI, but is not limited to such examples. Some or all of the above-described processing in the reading unit may be performed using AI or may be performed without using AI. For example, the reading unit can input facial expression data of the patient captured by a camera into the generative AI and have the generative AI perform the emotion estimation of the patient.

The formatting unit can estimate the patient's emotion and adjust the display method of the medical examination format based on the estimated emotion. For example, if the patient is nervous, the formatting unit provides a simple and highly visible format. If the patient is relaxed, the formatting unit can provide a format that includes detailed information. If the patient is anxious, the formatting unit can provide a format that focuses on key points. As a result, the formatting unit can adjust the display method of the medical examination format based on the patient's emotion. Emotion estimation is realized using an emotion engine or a generative AI with an emotion estimation function. The generative AI may be a text generation AI (e.g., LLM) or a multimodal generative AI, but is not limited to such examples. Some or all of the above-described processing in the formatting unit may be performed using AI or may be performed without using AI. For example, the formatting unit can input facial expression data of the patient captured by a camera into the generative AI and have the generative AI perform the emotion estimation of the patient.

The reception unit can analyze the patient's past recording history and select an optimal recording method. For example, the reception unit analyzes the patterns of videos previously recorded by the patient and starts recording in a similar manner. The reception unit can also evaluate the quality of videos previously recorded by the patient and propose optimal camera settings. The reception unit can also analyze the content of videos previously recorded by the patient and present necessary information in advance. As a result, the reception unit can analyze the patient's past recording history and select an optimal recording method. Some or all of the above-described processing in the reception unit may be performed using AI or may be performed without using AI. For example, the reception unit can input the past recording history into AI and have the AI select the optimal recording method.

The reception unit can filter the recording content based on the patient's current health condition or symptoms at the time of recording. For example, if the patient has a high fever, the reception unit instructs to record the display of a thermometer. If the patient complains of a rash, the reception unit can also instruct to focus on recording the area of the rash. If the patient complains of difficulty breathing, the reception unit can also instruct to record the breathing condition. As a result, the reception unit can filter the recording content based on the patient's current health condition or symptoms. Some or all of the above-described processing in the reception unit may be performed using AI or may be performed without using AI. For example, the reception unit can input the patient's health condition or symptoms into AI and have the AI perform the filtering of the recording content.

The analysis unit can apply different analysis algorithms based on the content of the video during analysis. For example, if the patient's symptoms are diverse, the analysis unit combines multiple analysis algorithms for analysis. If the patient's symptoms are concentrated in a specific area, the analysis unit can apply an analysis algorithm specialized for that area. If the patient's symptoms change over time, the analysis unit can apply a time-series analysis algorithm. As a result, the analysis unit can apply different analysis algorithms based on the content of the video. Some or all of the above-described processing in the analysis unit may be performed using AI or may be performed without using AI. For example, the analysis unit can input the content of the video into AI and have the AI select the optimal analysis algorithm.

The generation unit can adjust the level of detail of the summary based on the importance of the video when generating the summary. For example, if the video contains important symptoms, the generation unit generates a detailed summary. If the video contains minor symptoms, the generation unit can generate a concise summary. If the video contains highly urgent symptoms, the generation unit can generate a summary that can be quickly understood. As a result, the generation unit can adjust the level of detail of the summary based on the importance of the video. Some or all of the above-described processing in the generation unit may be performed using AI or may be performed without using AI. For example, the generation unit can input the importance of the video into AI and have the AI adjust the level of detail of the summary.

The reading unit can apply different reading algorithms based on the content of the summary when reading aloud. For example, if the summary contains important symptoms, the reading unit reads aloud in detail. If the summary contains minor symptoms, the reading unit can read aloud concisely. If the summary contains highly urgent symptoms, the reading unit can read aloud quickly. As a result, the reading unit can apply different reading algorithms based on the content of the summary. Some or all of the above-described processing in the reading unit may be performed using AI or may be performed without using AI. For example, the reading unit can input the content of the summary into AI and have the AI select the optimal reading algorithm.

The following is a brief description of the processing flow of Example 2 of the Embodiment.
Step 1: The reception unit records videos recorded by the patient or a related person. The videos recorded by the patient or a related person include, for example, videos in which the condition of the affected area or symptoms are described. The reception unit uses a smartphone or tablet to record the video and has a function to upload the recorded video to the cloud.
Step 2: The analysis unit analyzes the videos recorded by the reception unit. The analysis unit analyzes the content of the video and extracts symptoms and the condition of the affected area. The analysis unit can analyze the content of the video using AI.
Step 3: The generation unit generates a summary based on the videos analyzed by the analysis unit. The generation unit generates the summary using generative AI and summarizes the content of the video, converting it into a format that is easy for the doctor to understand.
Step 4: The reading unit reads aloud the summary generated by the generation unit. The reading unit can read aloud the summary by voice using AI.
Step 5: The formatting unit launches a medical examination format based on the summary generated by the generation unit. The formatting unit can automatically generate the medical examination format using AI.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the above-described reception unit, analysis unit, generation unit, reading unit, and formatting unit is realized by at least one of, for example, the smart device 14 and the data processing device 12. For example, the reception unit records a video of the patient using the camera 42 or microphone 38B of the smart device 14 and uploads it to the data processing device 12. The analysis unit analyzes the video using the specific processing unit 290 of the data processing device 12 and extracts the symptoms and condition of the affected area. The generation unit generates a summary using the specific processing unit 290 of the data processing device 12 and converts it into a format that is easy for the doctor to understand. The reading unit reads aloud the generated summary by voice using the control unit 46A of the smart device 14. The formatting unit automatically generates a medical examination format using the specific processing unit 290 of the data processing device 12. The correspondence between each unit and the device or control unit is not limited to the above examples and various modifications are possible.

### Second Embodiment

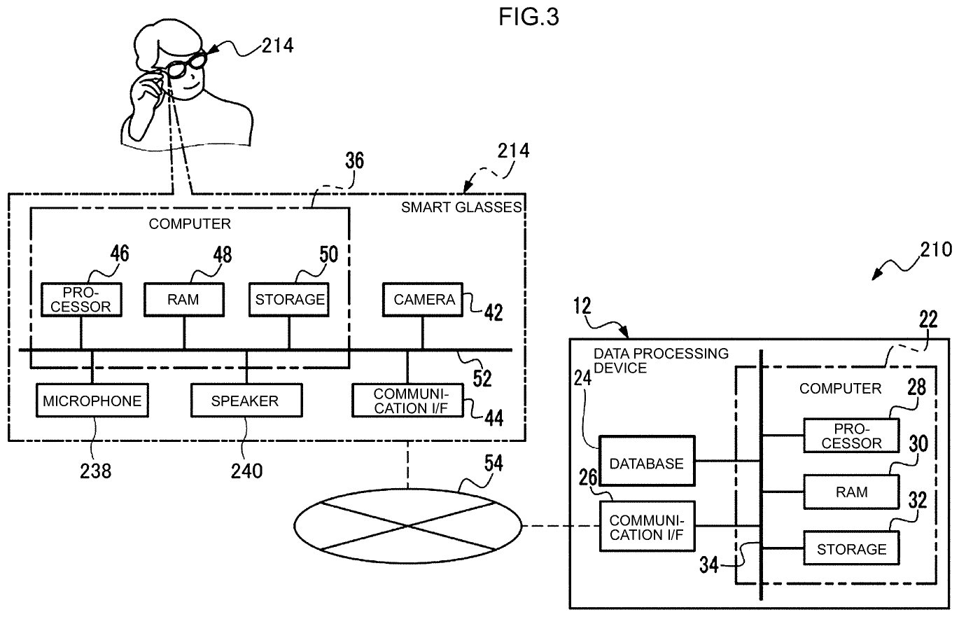

FIG. 3 shows an example configuration of a data processing system 210 according to the second embodiment.

As shown in FIG. 3, the data processing system 210 includes a data processing device 12 and smart glasses 214. An example of the data processing device 12 is a server.

The data processing device 12 includes a computer 22, a database 24, and a communication I/F 26. The computer 22 includes a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 includes a computer 36, a microphone 238, a speaker 240, a camera 42, and a communication I/F 44. The computer 36 includes a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

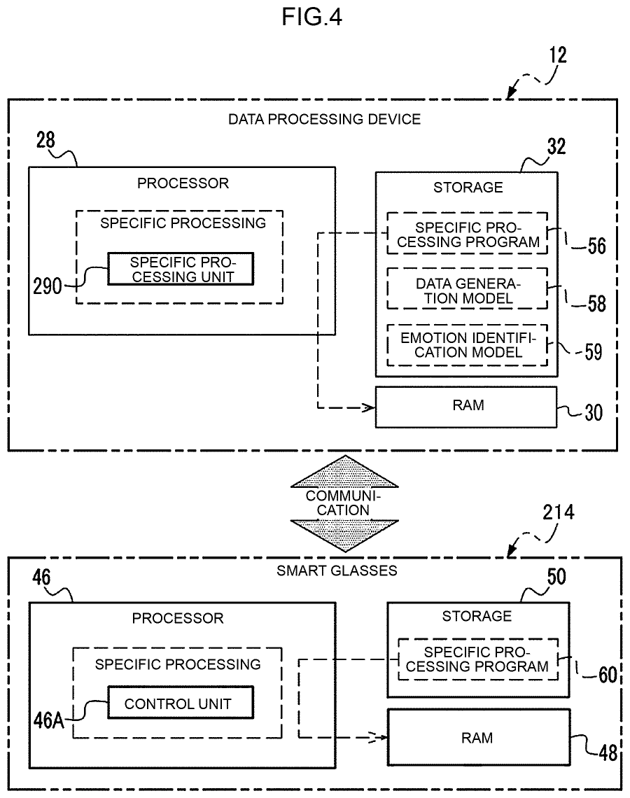

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the above-described reception unit, analysis unit, generation unit, reading unit, and formatting unit is realized by at least one of, for example, the smart glasses 214 and the data processing device 12. For example, the reception unit records a video of the patient using the camera 42 or microphone 238 of the smart glasses 214 and uploads it to the data processing device 12. The analysis unit analyzes the video using the specific processing unit 290 of the data processing device 12 and extracts the symptoms and condition of the affected area. The generation unit generates a summary using the specific processing unit 290 of the data processing device 12 and converts it into a format that is easy for the doctor to understand. The reading unit reads aloud the generated summary by voice using the control unit 46A of the smart glasses 214. The formatting unit automatically generates a medical examination format using the specific processing unit 290 of the data processing device 12. The correspondence between each unit and the device or control unit is not limited to the above examples and various modifications are possible.

### Third Embodiment

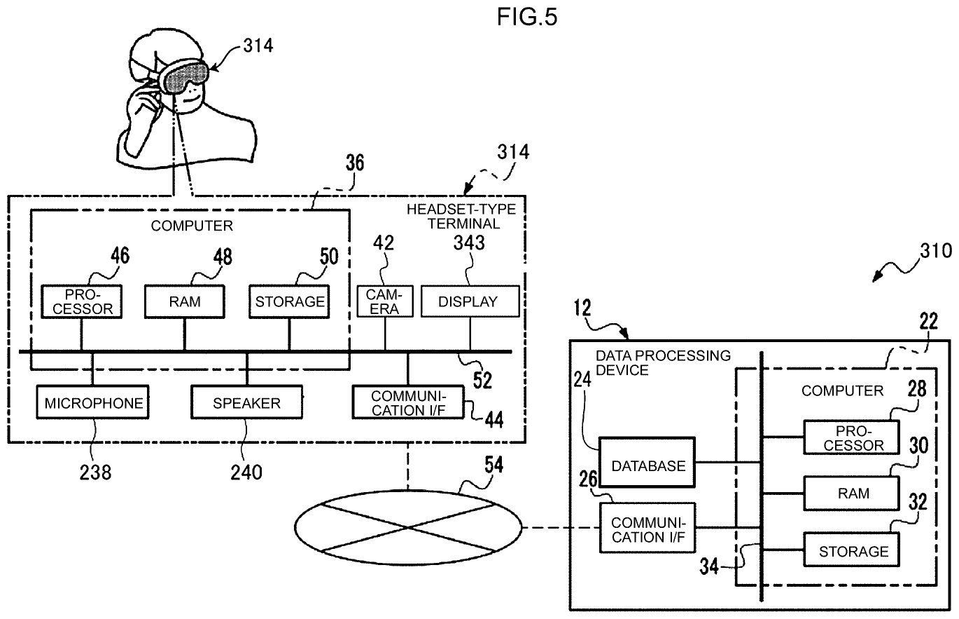

FIG. 5 shows an example configuration of a data processing system 310 according to the third embodiment.

As shown in FIG. 5, the data processing system 310 includes a data processing device 12 and a headset-type terminal 314. An example of the data processing device 12 is a server.

The data processing device 12 includes a computer 22, a database 24, and a communication I/F 26. The computer 22 includes a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 includes a computer 36, a microphone 238, a speaker 240, a camera 42, a communication I/F 44, and a display 343. The computer 36 includes a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

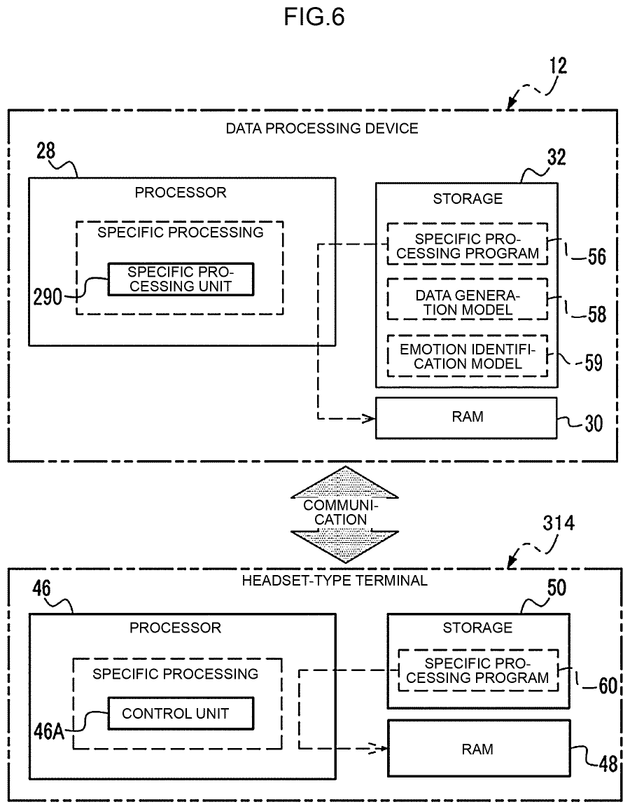

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the above-described reception unit, analysis unit, generation unit, reading unit, and formatting unit is realized by at least one of, for example, the headset-type terminal 314 and the data processing device 12. For example, the reception unit records a video of the patient using the camera 42 or microphone 238 of the headset-type terminal 314 and uploads it to the data processing device 12. The analysis unit analyzes the video using the specific processing unit 290 of the data processing device 12 and extracts the symptoms and condition of the affected area. The generation unit generates a summary using the specific processing unit 290 of the data processing device 12 and converts it into a format that is easy for the doctor to understand. The reading unit reads aloud the generated summary by voice using the control unit 46A of the headset-type terminal 314. The formatting unit automatically generates a medical examination format using the specific processing unit 290 of the data processing device 12. The correspondence between each unit and the device or control unit is not limited to the above examples and various modifications are possible.

### Fourth Embodiment

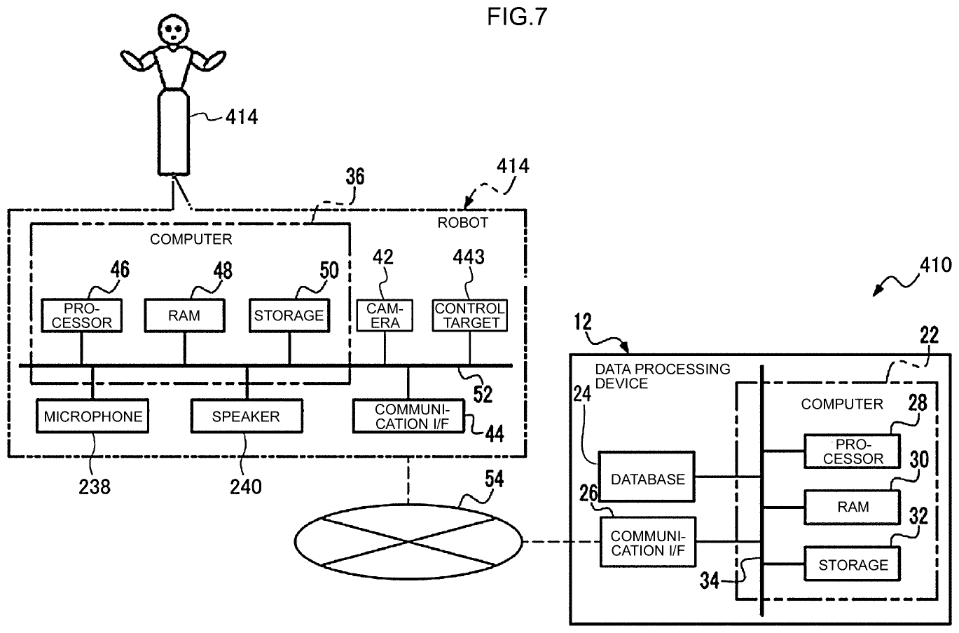

FIG. 7 shows an example configuration of a data processing system 410 according to the fourth embodiment.

As shown in FIG. 7, the data processing system 410 includes a data processing device 12 and a robot 414. An example of the data processing device 12 is a server.

The data processing device 12 includes a computer 22, a database 24, and a communication I/F 26. The computer 22 includes a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 includes a computer 36, a microphone 238, a speaker 240, a camera 42, a communication I/F 44, and a control target 443. The computer 36 includes a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

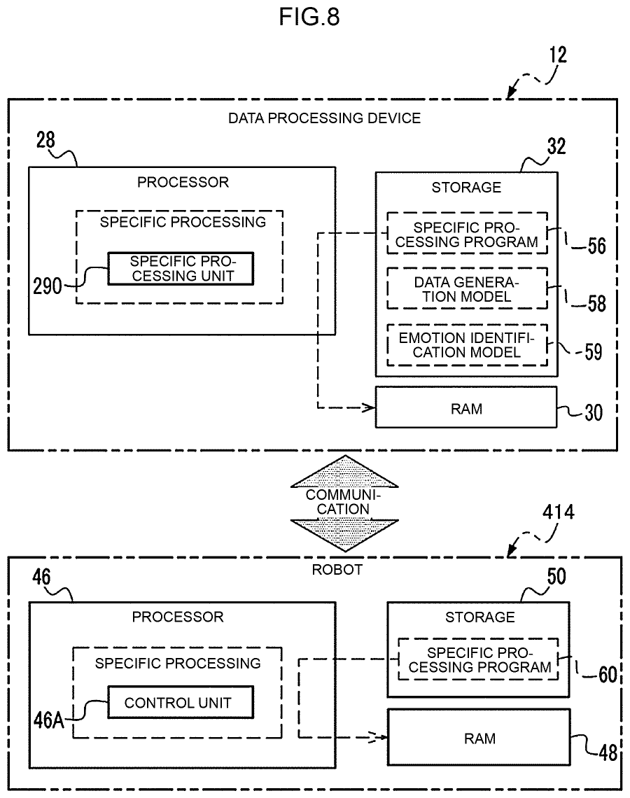

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the above-described reception unit, analysis unit, generation unit, reading unit, and formatting unit is realized by at least one of, for example, the robot 414 and the data processing device 12. For example, the reception unit records a video of the patient using the camera 42 or microphone 238 of the robot 414 and uploads it to the data processing device 12. The analysis unit analyzes the video using the specific processing unit 290 of the data processing device 12 and extracts the symptoms and condition of the affected area. The generation unit generates a summary using the specific processing unit 290 of the data processing device 12 and converts it into a format that is easy for the doctor to understand. The reading unit reads aloud the generated summary by voice using the control unit 46A of the robot 414. The formatting unit automatically generates a medical examination format using the specific processing unit 290 of the data processing device 12. The correspondence between each unit and the device or control unit is not limited to the above examples and various modifications are possible.

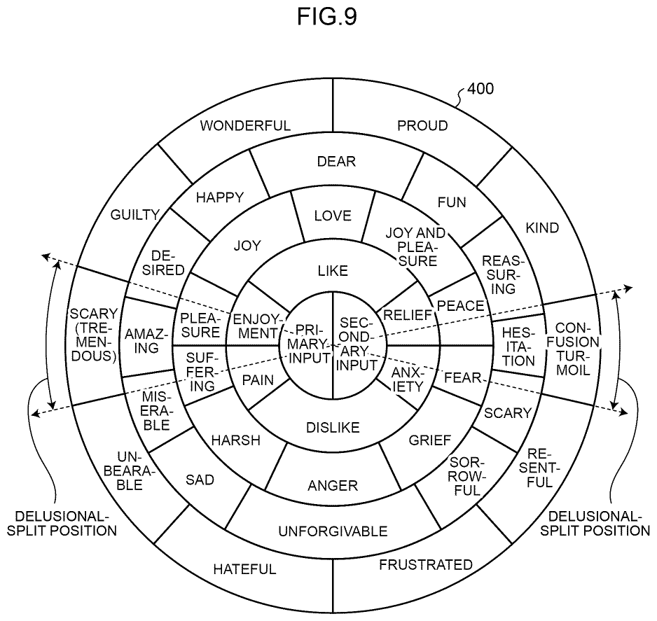

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

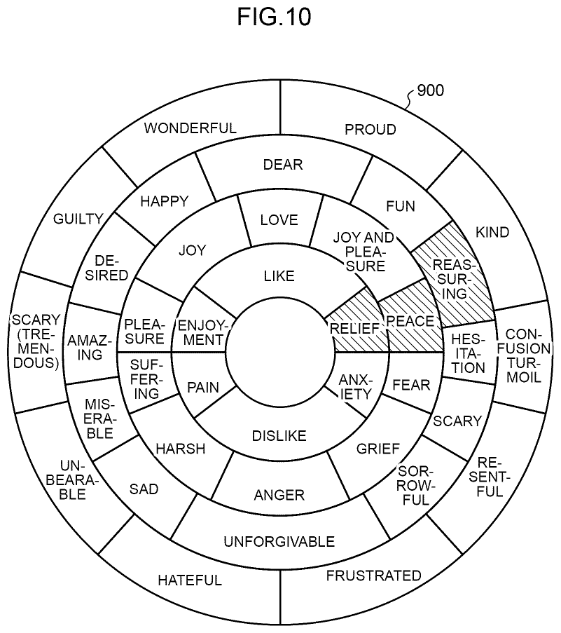

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used. Additionally, the examples described above were explained by dividing into form example 1 and form example 2, but these may be combined.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

[Additional Note 1] A system including: a reception unit configured to record a video; an analysis unit configured to analyze the video recorded by the reception unit; a generation unit configured to generate a summary based on the video analyzed by the analysis unit; a reading unit configured to read aloud the summary generated by the generation unit; and a formatting unit configured to launch a medical examination format based on the summary generated by the generation unit.

[Additional Note 2] The system according to Additional Note 1, wherein the reception unit is configured to record a video in which a patient or a related person talks about symptoms while recording the affected area.

[Additional Note 3] The system according to Additional Note 1, wherein the reception unit is configured to record items related to symptoms.

[Additional Note 4] The system according to Additional Note 1, wherein the reception unit is configured to record the condition of an injury.

[Additional Note 5] The system according to Additional Note 1, wherein the reception unit is configured to record the object of the injury.

[Additional Note 6] The system according to Additional Note 1, wherein the reading unit is configured to read aloud the generated summary.

[Additional Note 7] The system according to Additional Note 1, wherein the formatting unit is configured to launch a medical examination format before the patient's arrival via communication.

[Additional Note 8] The system according to Additional Note 1, wherein the reception unit is configured to estimate the patient's emotion and adjust the timing for starting the recording based on the estimated emotion.

[Additional Note 9] The system according to Additional Note 1, wherein the reception unit is configured to analyze the patient's past recording history and select an optimal recording method.

[Additional Note 10] The system according to Additional Note 1, wherein the reception unit is configured to filter the recording content based on the patient's current health condition or symptoms at the time of recording.

[Additional Note 11] The system according to Additional Note 1, wherein the reception unit is configured to estimate the patient's emotion and determine the priority of the content to be recorded based on the estimated emotion.

[Additional Note 12] The system according to Additional Note 1, wherein the reception unit is configured to prioritize recording content with high relevance by considering the patient's geographic location at the time of recording.

[Additional Note 13] The system according to Additional Note 1, wherein the reception unit is configured to analyze the patient's social media activity at the time of recording and record relevant content.

[Additional Note 14] The system according to Additional Note 1, wherein the analysis unit is configured to estimate the patient's emotion and adjust the analysis accuracy based on the estimated emotion.

[Additional Note 15] The system according to Additional Note 1, wherein the analysis unit is configured to apply different analysis algorithms based on the content of the video during analysis.

[Additional Note 16] The system according to Additional Note 1, wherein the analysis unit is configured to customize the analysis method according to the shooting environment or situation of the video during analysis.

[Additional Note 17] The system according to Additional Note 1, wherein the analysis unit is configured to estimate the patient's emotion and adjust the display method of the analysis result based on the estimated emotion.

[Additional Note 18] The system according to Additional Note 1, wherein the analysis unit is configured to determine the priority of analysis based on the shooting time of the video during analysis.

[Additional Note 19] The system according to Additional Note 1, wherein the analysis unit is configured to refer to related literature of the video during analysis to improve the accuracy of the analysis.

[Additional Note 20] The system according to Additional Note 1, wherein the generation unit is configured to estimate the patient's emotion and adjust the expression method of the summary based on the estimated emotion.

[Additional Note 21] The system according to Additional Note 1, wherein the generation unit is configured to adjust the level of detail of the summary based on the importance of the video when generating the summary.

[Additional Note 22] The system according to Additional Note 1, wherein the generation unit is configured to apply different summarization algorithms according to the category of the video when generating the summary.

[Additional Note 23] The system according to Additional Note 1, wherein the generation unit is configured to estimate the patient's emotion and adjust the length of the summary based on the estimated emotion.

[Additional Note 24] The system according to Additional Note 1, wherein the generation unit is configured to determine the priority of the summary based on the shooting time of the video when generating the summary.

[Additional Note 25] The system according to Additional Note 1, wherein the generation unit is configured to adjust the order of the summary based on the relevance of the video when generating the summary.

[Additional Note 26] The system according to Additional Note 1, wherein the reading unit is configured to estimate the patient's emotion and adjust the tone and speed of reading aloud based on the estimated emotion.

[Additional Note 27] The system according to Additional Note 1, wherein the reading unit is configured to apply different reading algorithms based on the content of the summary when reading aloud.

[Additional Note 28] The system according to Additional Note 1, wherein the reading unit is configured to adjust the level of detail of reading aloud according to the importance of the summary when reading aloud.

[Additional Note 29] The system according to Additional Note 1, wherein the reading unit is configured to estimate the patient's emotion and adjust the order of reading aloud based on the estimated emotion.

[Additional Note 30] The system according to Additional Note 1, wherein the reading unit is configured to determine the priority of reading aloud based on the shooting time of the summary when reading aloud.

[Additional Note 31] The system according to Additional Note 1, wherein the reading unit is configured to refer to related literature of the summary when reading aloud to improve the accuracy of reading aloud.

[Additional Note 32] The system according to Additional Note 1, wherein the formatting unit is configured to estimate the patient's emotion and adjust the display method of the medical examination format based on the estimated emotion.

[Additional Note 33] The system according to Additional Note 1, wherein the formatting unit is configured to apply different formatting algorithms based on the content of the summary when generating the medical examination format.

[Additional Note 34] The system according to Additional Note 1, wherein the formatting unit is configured to adjust the level of detail of the format according to the importance of the summary when generating the medical examination format.

[Additional Note 35] The system according to Additional Note 1, wherein the formatting unit is configured to estimate the patient's emotion and adjust the order of the medical examination format based on the estimated emotion.

[Additional Note 36] The system according to Additional Note 1, wherein the formatting unit is configured to determine the priority of the format based on the shooting time of the summary when generating the medical examination format.

[Additional Note 37] The system according to Additional Note 1, wherein the formatting unit is configured to refer to related literature of the summary when generating the medical examination format to improve the accuracy of the format.

What is claimed is:

1. A system comprising: a reception unit configured to record a video; an analysis unit configured to analyze the video recorded by the reception unit; a generation unit configured to generate a summary based on the video analyzed by the analysis unit; a reading unit configured to read aloud the summary generated by the generation unit; and a formatting unit configured to launch a medical examination format based on the summary generated by the generation unit.

2. The system according to claim 1, wherein the reception unit is configured to record a video in which a patient or a related person talks about symptoms while recording the affected area.

3. The system according to claim 1, wherein the reception unit is configured to record items related to symptoms.

4. The system according to claim 1, wherein the reception unit is configured to record the condition of an injury.

5. The system according to claim 1, wherein the reception unit is configured to record the object of the injury.

6. The system according to claim 1, wherein the reading unit is configured to read aloud the generated summary.

7. The system according to claim 1, wherein the formatting unit is configured to launch a medical examination format before the patient's arrival via communication.

8. The system according to claim 1, wherein the reception unit is configured to estimate the patient's emotion and adjust the timing for starting the recording based on the estimated emotion.
